(19) United States
(12) Patent Application Publication
(10) Pub. No.: US 2011/0177165 A1
(43) Pub. Date: Jul. 21, 2011

(54) MULTIPARTICULATE TABLET AND METHOD FOR THE PRODUCTION THEREOF

(75) Inventors: Frédéric Gerber, Blotzheim (FR); Burkhard Schluetermann, Au (DE)

(73) Assignee: Add Technologies Ltd., Reinach (CH)

(21) Appl. No.: 12/998,180

(22) PCT Filed: Sep. 16, 2009
(86) PCT No.: PCT/EP2009/006707

§ 371 Date: Mar. 24, 2011

(30) Foreign Application Priority Data
Sep. 24, 2008 (DE) 10 2008 048 729.5

(51) Int. Cl. A61K 9/22; A61K 31/60; A61K 31/192; A61P 25/00

(52) U.S. Cl. 424/465; 514/165; 514/570

(57) ABSTRACT

A tablet, containing

an inner phase, comprising
a) a first type of pellets (I) which contain a first active ingredient and which have a coating and/or release the active ingredient in a modified manner, or
b) a first type of coated first active ingredient (I),
   mixed with
c) spherical particles (II),
   and an outer phase containing one or more excipients present in non-granulated form before tableting, selected from disintegrants, lubricants, flow regulators, fillers and binders,
   wherein the weight of the outer phase makes up not more than 25 percent of the total weight of the tablet.

The invention relates to multiparticulate tablets and methods for the production thereof. The invention furthermore relates to a granulation method for producing spherical particles for use in the multiparticulate tablets and other multiparticulate administration forms.

In multiparticulate (multiple-unit) pharmaceutical forms, the dose of one or more active ingredient is divided into many (generally more than a thousand subunits), which are formed by the respective particles containing one or more active ingredients. These subunits or particles can be, for example, pellets or spherical particles. These typically have either a layer-like structure or a matrix structure.

Both layered and matrix pellets can in each case be finally coated with one or more films. Coating with a film can be tracked, if necessary, for various purposes, for instance for improving the processability, for example by improving the flow behavior or decreasing the hygroscopicity, for guaranteeing and improving the chemical and/or mechanical stability of the pharmaceutical, for masking a bad taste or a disagreeable odor or for changing the solution or release rate of the incorporated active ingredient(s).

The multiparticulate administration forms are particularly of interest in modified-release systems, since they especially have advantages compared to the “single-unit” pharmaceutical forms for biopharmaceutical reasons. Single units are monolithic pharmaceutical forms, i.e. modified-release individual pharmaceutical forms, which pass through the gastrointestinal tract undisintegrated, become ever smaller due to degradation or erosion or only release the pharmaceuticals in the intestines (R. Voigt, Pharmazeutische Technologie, [Pharmaceutical Technology], 8th edition, Berlin, 1995). In contrast to this, the multiparticulate systems disintegrate into their subunits, which in each case can exhibit a desired controlled release behavior. Damage to the tablet or a malfunction with respect to release therefore customarily has a markedly less negative effect, if at all, in the case of multiparticulate pharmaceutical forms than in, for example, a film-coated or alternatively matrix form of a single-unit preparation.

Among the pharmaceutically employed administration forms, oral preparations still have the greatest importance. For the patient, these are the most pleasant and most customary pharmaceutical preparations. Multiparticulate pharmaceutical forms can be filled into capsules, e.g. hard gelatin capsules, or into sachets for administration, or, which from pharmaceutical points of view is the most demanding, can be present compressed to give tablets. Often, especially in the case of tablets, an additional film is applied for facilitating swallowability, or in order to improve the appearance of such administration forms. The most common pharmaceutical form among these solid medicaments is the tablet, which compared to a hard gelatin capsule not only tends to have an improved swallowability, but also can be produced more economically.

The compression of multiparticulate systems, especially of coated multiparticulate systems, however, is not simple, since many factors can also mutually influence the quality of the pharmaceutical form. For example, thin films are in general more rapidly releasing and are, however, also more easily destroyed by the press force applied in the case of subsequent tableting to give tablets.

Important factors that play a role in the production of a qualitatively high-quality multiparticulate administration form are, in addition to a) the selection of suitable pharmaceutical excipients, and b) the choice of suitable, well-controlled production methods, also c) the structure of a tablet.

In principle, the composition of a multiparticulate tablet corresponds essentially to the structure of a conventional tablet formulation for immediate release described below:

This consists a) of an inner phase and b) an outer phase.

The inner phase, also called internal phase, of a tablet contains the active ingredient, usually in granulated, i.e. aggregated, form. As excipients, the inner phase typically contains fillers, binders and, if necessary, disintegrants and/or other excipients as main constituents.

The outer phase of a tablet, often designated as the external phase, typically contains, for example, disintegrants, lubricants and flow-regulating agents in finely powdered and non-aggregated form, but possibly also small amounts of fillers and binders. Additionally, flavorings and further additives can be present. The term outer phase is to be understood functionally, not spatially.

A good survey of customary excipients is found in H. P. Fiedler, Lexikon der Hilfsstoffe für Pharmazie, Kosmetik and angrenzende Gebiete [Encyclopedia of Excipients for Pharmacy, Cosmetics and neighboring Fields], Editio Cantor Verlag Aulendorf.

In a conventional tablet formulation, as a rule of thumb the ratio between inner phase and outer phase, with respect to optimum flow properties and packing densities, is approximately 8:2 and 9:1 (K. H. Bauer, K.-H. Frömming, C. Führer, Pharmazeutische Technologies [Pharmaceutical Technologies], Thieme, 1986).

In a multiparticulate, solid pharmaceutical form, the phase ratio is generally different. Here, the content of the outer phase is markedly increased. In acceptable, multiparticulate, high-dose preparations, the phase ratio of inner to outer phase should be in the range 3:7, but at least 4:6, if not higher (M. Braun, dissertation, 2003, Rheinische-Friedrich-Wilhelms University Bonn; K. G. Wagner, dissertation, 1999, Eberhard-Karls University, Tübingen).

It is of particular importance here that the inner phase of the tablet formulation, which generally contains the active ingredient-containing particles, is protected against the action of force during tableting by the proportionately increased outer phase. As a result of the action of force or the mechanical stress resulting therefrom, a change in the product characteristics can result. This especially applies in the case of the multiparticulate particle systems described here, which are characterized in that the product characteristics of this administration form are defined at the stage of the respective subunits and if possible these subunits should not be influenced disadvantageously by tableting.

In order to achieve this, the phase ratio of inner to outer phase in such administration forms is therefore generally increased in favor of the outer phase, in order, inter alia, still to protect pellets against damage by the mechanical stress during tableting with adequate weight and content uniformity of the administration form. Tableting can cause a change in the product characteristics.

Administration forms in which the particles are in each case coated with a film or functional lacquer, especially contain, in contrast to the aforementioned conventional tablets, a markedly increased content of outer phase, in order to reduce damage to the film of a coated pellet as a result of the action of force during tableting, in order not to change the product characteristics of the incorporated pellets or coated pharmaceuticals and to guarantee an adequate mechanical stability of the tablet, an acceptable release behavior, but also an adequate weight and content uniformity. The requirements of the content uniformity of an administration form is particularly critical in the case of the multiparticulate administration forms. Testing for the uniformity of the content and the requirements of administration forms are adequately documented in the current pharmacopeias.

The processing of coated active ingredients is similarly critical as that of the pellets described beforehand. Therefore, within the meaning of this specification coated active ingredients that are subsequently processed to give a tablet are also a subject of the invention further explained here.

The proportion of outer phase can thus in these cases be up to 70% or even more, the most frequently employed excipient being fibrous, microcrystalline cellulose, in which pellets or coated active ingredients can be embedded particularly stably.

As a result of the action of force during tableting, for example, the release characteristics of the pellets in a tablet can be altered disadvantageously, so that the release of the pharmaceutical, for example, is accelerated, gastric juice resistance is lost, or an existing masking of taste is completely abolished. These disadvantages are all the more critical if the pharmaceutical doses to be administered are high, if profiles with different release rates or type of release are to be combined and/or if a number of pharmaceuticals are to be combined with one another to give an administration form. The latter preparations are called combination preparations—or alternatively fixed combinations—medicaments containing a number of active ingredients.

A combination of a number of active ingredients can especially be sensible if it is demonstrated that each individual active ingredient is therapeutically active in relation to the claimed field of use and the dose of each individual ingredient is calculated with respect to the highest dose, the administration frequency and duration such that an appreciable number of patients needs such a fixed combination and it is efficacious and harmless in terms of a benefit and risk ratio, and the active ingredients administered increase the efficacy and/or harmlessness of the active ingredients or of the main active ingredient or decrease the possibility of abuse of the main ingredient or the fixed combination of active ingredients produces a greater therapeutic effect or offers greater harmlessness than any individual active ingredient separately. These criteria, which are known as Crout's criteria, are also taken into consideration in pharmaceutical law.

Thus there are many examples of very expedient combination preparations that contain two or more pharmaceuticals that, inter alia, assist one another in their action. It is especially difficult for elderly people to get used to the taking of various medicaments. The taking of a combination preparation facilitates and in many cases guarantees the treatment, inter alia even in pediatrics, and thus improves compliance, and contributes to pharmaceutical safety.

Combination preparations are very often employed, inter alia, in the treatment of high blood pressure, for the treatment of Parkinson's disease, for the treatment of diseases of the central nervous system, for infection defense with antibiotics or antivirally active substances, for oral contraception, for the treatment of gastric disorders and in pain therapy.

The mass and dimensions of combination preparations or generally also of multiparticulate administration forms customarily sometimes differ from the correspond administration forms of the individual pharmaceutical and its rapid-release administration forms if very low-dose preparations, for instance the hormone preparations for contraception, are disregarded.

This especially applies if the pharmaceutical or a number of pharmaceuticals of a pharmaceutical combination product is (are) relatively high-dose or the dissolution rate of one or more active ingredients is to be modified. This also applies, however, for products with taste-masked active ingredients or pellets that have coatings for masking taste and are subsequently to be processed to give tablets, especially to give orally disintegrating tablets.

Up to now, in addition to coated pellets or coated active ingredients, it was impossible or very difficult to process further pharmaceuticals in high-dose form simultaneously to give combination products, since with respect to mass and dimensions tablets that were simple to administer, that is swallowable, no longer resulted or the product characteristics of the individual pellet types changed unacceptably.

It is therefore an object of the present invention to provide a multiparticulate tablet containing a decreased amount of outer phase. It is a further object of the present invention to provide a multiparticulate tablet containing a combination of active ingredients. A further object of the present invention is to provide a multiparticulate tablet containing a comparatively high active ingredient content.

Moreover, difficulties exist in the production of multiple-unit pharmaceutical forms, especially if, for example, fixed combinations are to be formulated as multiparticulate administration forms.

Previously known granulation methods are frequently not satisfactory, since a high-dose active ingredient must be overdiluted with suitable excipients for its processing or inadequate process technologies are available in order to obtain acceptable properties of intermediate and final products with respect to processability, size, mass, disintegration, hardness and release.

Known granulation methods moreover have the disadvantage that active ingredients that react very sensitively to moist and dry aggregation methods on use of a small quantity of additional excipients, for instance fillers, binders, disintegrants, flow regulators and lubricants (e.g. <60% by weight) only release the active ingredient from the corresponding granules or final administration forms inadequately and/or not very controllably. By means of such granulation methods, highly compacted agglomerates frequently result, which subsequently only inadequately release the active ingredient. Examples thereof are, for example, ascorbic acid and oxcarbazepine.

The typical granulation method for a pharmaceutical is complex. After mixing, aggregating by moistening, kneading, heating or pressure, and drying following a moist granulation, the granules obtained must be comminuted again and classified according to their requirements.

It was therefore a further object of the present invention to make available a readily controllable granulation method and corresponding formulations even for high-dose active ingredients in combination with pellet products or coated active ingredients (I) containing a high content of active ingredient in order at the same time to make possible good processing, disintegration and dissolution characteristics of all pharmaceutically active constituents of an administration form, which during production essentially leave unchanged the characteristics of the incorporated pellets (I), especially those of dissolution.

The aforementioned objects are achieved by tablets and methods according to the invention. Advantageous embodiments are described below and are the subject of the dependent claims.

According to a first aspect, the invention now relates to a tablet, containing an inner phase, comprising

a) a first type of pellets (I), which contain a first active ingredient and which have a coating and/or release the active ingredient in a modified manner, or
b) a first type of coated first active ingredient (I), mixed with
c) spherical particles (II),
and an outer phase containing one or more excipients present before tableting in nongranulated form, selected from disintegrants, lubricants, flow-regulating agents, fillers and binders,
the weight of the outer phase making up not more than 25 percent of the total weight of the tablet, for example not more than 20%, 15%, 10% or 5%.

The weight of the outer phase can make up, for example, up to 25%, 22.5%, 20%, 17.5%, 15%, 12.5%, 10%, 7.5%, 5%, 4%, 3%, 2% or 1% of the total weight of the tablet. The weight of the outer phase can make up, for example, at least 0.5%, 1%, 2%, 3% or 5% of the total weight of the tablet. The excipients used for the outer phase present in non-granulated form before tableting can be suitable customary excipients (see H. P. Fiedler, Lexikon der Hilfsstoffe fur Pharmazie, Kosmetik and angrenzende Gebiete [Encyclopedia of Excipients for Pharmacy, Cosmetics and Neighboring Fields], Editio Cantor Verlag Aulendorf). Additionally, flavorings, colorants, solubilizers and further additives can be contained in the outer phase.

The weight of the first type of pellets (I) or the first type of the coated first active ingredient (I) can make up, for example, 35 to 55% of the total weight of the tablet, for example from 37.5 to 52.5% or from 40 to 50%. If more types of pellets (I) or coated active ingredients (I) are used, this accordingly applies for their total weight.

The weight of the spherical particles (II) in the embodiments, which moreover contain at least one first type of pellets (I) and/or coated active ingredient (I) can be, for example, in the range from 30% to 70%, preferably in the range from 35 to 65%, or 40 to 60%, of the total weight of the tablet.

Thus multiparticulate tablets are produced whose inner phase contains a first pellet product or coated active ingredient (I) and whose outer phase, compared to a conventional multiparticulate administration forms, can be markedly reduced by admixing spherical particles (II) to the first pellet product (I) or to the coated active ingredient.

Generally, the outer phase of a multiple-unit tablet must absorb kinetic energy that reaches the product through tableting. It therefore significantly contributes to the fact that mechanically adequately stable tablets result. For the quality of the outer phase of a multiparticulate tablet, which is to be produced under as gentle as possible conditions, a balanced ratio of elastic and plastic deform-ability is necessary.

The spherical particles (II), which are common to all embodiments of this invention, have advantageous characteristics, especially a balanced ratio of elastic and plastic characteristics, which surprisingly make possible a saving of outer phase. In addition to the compression characteristics of these particles, their technological characteristics, such as shape, density and porosity, are likewise important for the substitution of the outer phase for multiparticulate tablets.

A preferred production method and the advantageous characteristics of the spherical aggregates (II) are described in more detail below.

The spherical particles (II) that are added to the pellets (I) or coated active ingredients (I) can consist solely of (pharmaceutical) excipients. Examples of excipients that can be employed advantageously are described below in connection with a preferred granulation method and in the context of the exemplary embodiments.

The pellets (I) and the spherical particles (II) differ from one another with respect to at least one parameter, for example with respect to their composition, their structure and/or with respect to a physical parameter that can be influenced, for example, by the production, such as, for example, density, porosity etc.

The pellets (I) are preferably spherical pellets. The pellets (I) can be produced according to the same method as the spherical particles (II); the composition then contains two types of spherical products or particles (II). They can also be prepared, however, by another method.

The first type of pellets is constituted in particularly preferred embodiments such that the active ingredient contained in it is released in a modified manner.

The pellets used according to the invention can have a coating, for example a film coating. The coating, however, can have the customary functions for such coatings. The coating, for example, can be a coating for taste masking, a coating for odor masking, a coating for stabilization of the active ingredient, a coating for improving the processability, a coating for improving the flow behavior, a coating for decreasing the hygroscopicity or a coating for guaranteeing and improving the chemical and/or mechanical stability of the pharmaceutical.

Particularly preferably, at least the first type of pellets (I) is provided with a coating which modifies the release of the active ingredient from the pellets (I). Modification of the release comprises, for example, uniformly prolonged active ingredient release, extended active ingredient release, delayed active ingredient release, stepped active ingredient release and combinations thereof (controlled release, extended release, prolonged release, repeated release, delayed release).

Examples of materials that are suitable for coatings of this type comprise cellulose derivatives, such as, for example, ethylcellulose, methylcellulose, hydroxypropylmethyl cellulose, cellulose acetate phthalate, polymers of methacrylic acid and methacrylic acid esters, for instance Eudragit, or polyvinyl derivatives,

Alternatively, instead of by a coating, a modification of the release from the pellets can also be achieved by other measures customary in the field, for example by constructing the pellets as a matrix, which allows a modification of the release.

Particularly advantageous embodiments of tablets of the present invention are generally those in which the pellets (I) are coated with a film, i.e. the coating is present as a film.

According to the above details for the coatings, they can be, for example, a taste-masking film or an odor-masking film. Even more advantageous are those embodiments in which the pellets (I) are coated with a film which modifies the release of the active ingredient (or of the active ingredients) from the pellet (I). These can be the customary films or functional lacquers known in the field. Possible functions of such films or functional lacquers are, for example, the delaying of active ingredient release and the production of gastric juice resistance. The film coating can be, for example, an enteric coating.

The same applies for the coating of the coated active ingredients; the coating can especially perform the same functions as the coating of the pellets, for example modify the release. Reference is therefore made to the above-mentioned examples for coatings.

Coatings, coverings and films are typically active ingredient-free. However, embodiments are also conceivable in which active ingredient is contained in the coating.

A first type of pellets (I) having a first film covering or a first coating and a second type of pellets (I) having a second film covering or a second coating can be employed in exemplary embodiments, the two types of pellets (I) containing the same active ingredient, but being different from one another with respect to their release behavior. A pellet type can be coated, for example, with a film modifying, especially slowing or delaying, the release, while the other pellet type releases the active ingredient immediately. An advantageous duration and continuity of active ingredient release can thereby be achieved.

Embodiments are also conceivable in which the first type of pellets containing the first active ingredient is present in combination with a first type of coated first active ingredient. In such embodiments, for example, the first type of pellets and the first type of covered active ingredient can exhibit a different release behavior (dissolution profile) of this same active ingredient. For example, the first type of pellets, as already mentioned, can be covered with a film influencing, especially slowing, release, while the covered active ingredient releases the active ingredient immediately. Alternatively, the covering of the active ingredient can slow the release.

Embodiments are furthermore conceivable in which, in addition to the first type of pellets (I), a second type of pellets containing a second active ingredient is present. Thus combination preparations can be realized in which on account of the decreased need of outer phase the tablet dimensions are decreased and thus the swallowability can be improved. Here too, various coatings or film coverings can be used. By suitable selection of films or pellet construction or composition, active ingredients otherwise incompatible with one another can also be realized in one tablet as a combination preparation.

In all these variants, the spherical particles can be active ingredient-free. Alternatively, the spherical particles (I) can also contain one or more active ingredients. Particularly advantageous combination preparations can be obtained thereby. The spherical aggregates (II) can be uncoated or likewise provided with a coating and the above explanations for possible coatings of the pellets (I) analogously apply here.

For example, embodiments are conceivable in which the first type of pellets (I) and the spherical particles (II) contain the same active ingredient, but have different release profiles. For example, the first type of pellet (I) can be coated with a film modifying, especially slowing and/or delaying, the release, while the spherical particles (II) immediately release the active compound. An advantageous duration and continuity of the active ingredient release can thereby be achieved.

The spherical particles can also contain a second active ingredient or a number of active ingredients which, for example, is (are) different from the first active ingredient of the pellets (I) or of the covered active ingredient (crystals).

Combination preparations can thus be produced particularly advantageously. A preferred embodiment thus relates to a tablet which contains:

an inner phase, comprising
a) a first type of pellets (I), which contain a first active ingredient and have a coating and/or release the first active ingredient in a modified manner, or
b) a first type of covered first active ingredient (I), mixed with
c) spherical particles (II), which contain a second active ingredient,
and an outer phase containing one or more excipients present before tableting in non-granulated form, selected from disintegrants, lubricants, flow regulators, fillers and binders,
the weight of the outer phase making up not more than 25 percent of the total weight of the tablet, for example not more than 20%, 17.5%, 15%, 12.5%, 10%, 7.5% or 5%.

Analogously to the explanations given above, the spherical particles can also be coated or covered with film, and various release profiles can be combined with one another, for instance pellets for immediate release containing spherical aggregates, which are coated with a film modifying the release, or conversely.

The spherical particles (II) are preferably produced by moist granulation, particularly preferably by granulation in a fluidized bed. The spherical particles can especially be produced by rotor granulation in a fluidized bed, preferably by means of a granulation method with directed material movement and particularly preferably by rotor granulation using a bladed rotor.

An example of a suitable method for the production of these round granules or spherical aggregates (II), which can exhibit a high content of active ingredient, is a granulation method, in which

a) one or more excipients and one or more active ingredients are mixed and premoistened with a liquid in a vertical granulator,
b) the moistened mixture is subsequently processed in a bladed rotor with liquid addition to give an essentially spherical aggregate,
c) is dried in a fluidized bed unit or another suitable machine and
d) subsequently, after possibly necessary separation of coarse grain, and after addition of one or more excipients and a first type of pellets (I) or of a first type of coated active ingredient (I), can be processed to give a suitable pharmaceutical intermediate or final product.

A suitable apparatus and a suitable method are fundamentally described in International Patent Application WO 2004/052607 A1. It was found that thereby when using special method conditions, spherical particles, especially with a high active ingredient loading, can be prepared, which are advantageously used in the present invention and surprisingly make possible a saving of outer phase, even at high active ingredient contents.

According to a preferred embodiment, the spherical aggregates are prepared as follows:

(a) introduction of the starting material powder, which is optionally wetted with a pharmaceutically suitable liquid diluent, in an apparatus that contains:
a rotor chamber having an axially extending cylindrical wall,
a device to lead air through the rotor chamber from the bottom,
a spray device for the supply of liquid to the chamber, one or more inlet openings for introducing the powder mixture,
a rotor that rotates around a vertical rotor axis, the rotor being arranged in the rotor chamber, having a central horizontal surface and in at least the external third of the rotor, having the shape of a conical shell having an outward- and upward-directed inclination of between 10° and 80°, the conical shell having a circular upper edge that lies in a plane that is perpendicular to the rotor axis,
a multiplicity of stator blades in each case having an external end that is statically fixed to the cylindrical wall of the rotor chamber above the plane that is formed by the upper edge of the conical shell of the rotor, and an internal end that extends into the rotor chamber and is arranged tangentially to the cylindrical wall of the rotor chamber and in the cross-section to the rotor axis essentially has the shape of an arc of a circle or a spiral,
(b) rotation of the rotor so that the product, which is circulated by kinetic energy for an adequate period of time, moves from the rotor to the internal surface of the stator blades before it falls back onto the rotor, optionally while air is supplied and/or a pharmaceutically acceptable liquid is sprayed into the rotor chamber so that solid pellets having a desired diameter are formed.

According to an analogous method, correspondingly advantageous spherical particles (II) without active ingredient can also be produced.

Active ingredients that when using a small quantity of additional excipients react very sensitively in conventional methods to moist and dry aggregation methods so that the active ingredient would only be inadequately and/or not very controllably released from the corresponding granules or final administration forms, can especially be converted successfully with the aid of the above method into spherical particles, which even allow the excipient content in the tablet to be reduced by ⅓ or ½.

The excipient(s) mentioned in step a) can comprise, for example, one or more granulation-promoting excipient(s).

According to the preferred production method mentioned beforehand, the spherical particles (II) preferably consist of one or more suitable (pharmaceutical) excipients, to which one or more active ingredients are added. (Pharmaceutically) suitable excipients used here are especially water-soluble, water-swellable but also water-insoluble excipients from the group consisting of the fillers and binders and disintegration-promoting excipients (disintegrants).

Preferably, the filler is a fine, microcrystalline cellulose and/or a water-soluble carbohydrate, for instance mannitol, sorbitol or xylitol, the binder is a water-soluble, polymeric excipient such as polyvinylpyrrolidone, hydroxypropylmethylcellulose, hydroxypropylcellulose, methylcellulose, hydroxymethylcellulose, hydroxyethylcellulose, hydroxypropylcellulose, alginates, pectins, polyvinyl acetates, xanthans, and other binders that can customarily be used for tablet production, and mixtures thereof.

The disintegration-promoting excipient or disintegrant is preferably selected from crosslinked polyvinylpyrrolidone, crosslinked sodium carboxymethylcellulose and crosslinked sodium carboxymethylstarch, since these excipients, in addition to their disintegrating action, also facilitate aggregation to give spherical bodies, since they act during production as a moisture buffer and thus stabilize the production method.

The moistening and granulation agent employed during the pre-moistening and aggregation in the bladed rotor is preferably water. In further preferred variants, the moistening agents employed are organic liquids such as alcohols or ketones, for instance methanol, ethanol, isopropanol and acetone, or mixtures of alcohols and water or ketones and water. In a further preferred variant, the binder can also be added in dissolved form to the moistening agents.

The cumulative active ingredient content of the spherical particles (II) prepared therefrom can be very high and is, for example, up to 95%. It is thus advantageously possible using the granulation method described to produce spherical particles (II), especially for high-dose pharmaceuticals, which can subsequently be combined with pellets (I) or covered active ingredients (I) to give a multiparticulate pharmaceutical form.

Surprisingly, however, it has also been found that these spherical granules or particles (II) can be compressed to give tablets after admixture of only a small quantity of outer phase, i.e. excipients present in non-granulated form before tableting, as were described above. This is a special working variant of this invention.

According to a further aspect of the present invention, a tablet is accordingly provided which contains:

an inner phase, comprising spherical particles (II), and an outer phase containing one or more excipients present in non-granulated form before tableting, selected from disintegrants, lubricants, flow-regulating agents, fillers and binders,
the weight of the outer phase making up not more than 25 percent of the total weight of the tablet, for example not more than 20%, 17.5%, 15%, 12.5%, 10%, 7.5% or 5%.

In these embodiments, the weight of the spherical particles (II) containing active ingredient can make up, for example, between 75 and 95%, e.g. at least 77.5%, at least 80%, at least 85% or at least 90% of the total weight of the tablet.

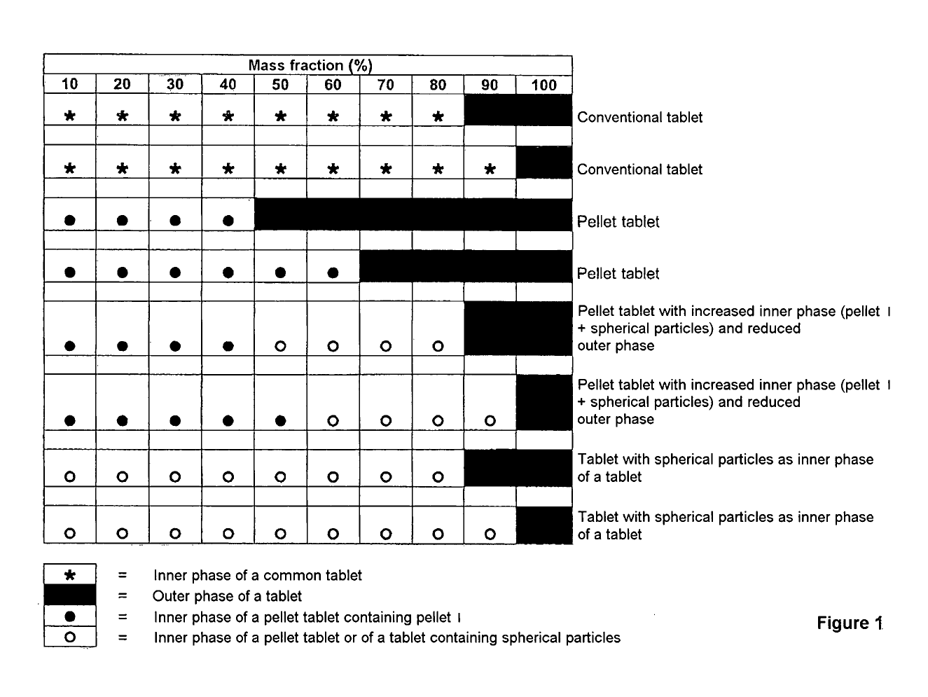

A graphic presentation of phase ratios in tablets is found in FIG. 1. FIG. 1 shows the percentage mass fractions of the inner and outer phase of conventional, multiparticulate tablets and of tablets for the purposes of this invention.

A content of outer phase that makes up not more than 25 percent of the total weight of the tablet, for example not more than 20%, 17.5%, 15%, 12.5%, 10%, 7.5% or 5% thus results.

It was also surprisingly found that the spherical particles (II) can be prepared, for example, using the special method described beforehand such that after tableting the product characteristics of the first pellet product (I) or those of the covered active ingredient (I) are only changed very little or virtually unchanged and the administration form prepared therefrom has acceptable product characteristics. Thus the show differences in the release of pellets (I) after tableting with spherical particles (II) for the purposes of the invention of less than 20, even less than 15, and especially less than 10%.

According to a further aspect of the invention, a tablet is provided which contains:

an inner phase, comprising
a) a first type of pellets (I) which contain a first active ingredient and which have a coating and/or modify the release of the active ingredient, or
b) a first type of covered first active ingredient (I), mixed with
c) spherical particles (II), which were preferably produced using moist granulation methods, especially fluidized bed granulation methods, but also rotor methods in the fluidized bed, and especially a bladed rotor,
the difference in the dissolution profiles of the first type of pellets (I) or the first type of covered active ingredient (I) after admixing the spherical aggregate (II) and subsequent tableting being less than 20%, preferably less than 15%, but also less than 10%.

In other words, the profile of the dissolution of the first active ingredient from the first type of pellets (I) or the first type of covered active ingredient (I), i.e. before admixing the spherical particles (II) and before subsequent tableting, differs in this aspect of the invention by less than 20%, preferably less than 15%, and most preferably less than 10% from the profile of the dissolution of the first active ingredient from the tablet according to the invention.

In this embodiment too, the spherical particles (II) can contain one active ingredient or a number of active ingredients, which preferably is/are different from the first active ingredient of the pellets (I) or of the covered active ingredient (I). An advantageous embodiment consequently relates to a tablet that contains:

a first type of pellets (I) which contain a first active ingredient and which have a coating and/or release the active ingredient in a modified manner, or a first type of coated first active ingredient (I),
mixed with spherical particles (II), which were produced using a bladed rotor and contain a second active ingredient, the difference in the dissolution profiles of the first type of pellets (I) after admixing the spherical aggregate (II) and subsequent tableting being less than 20%, preferably less than 15%, but also less than 10%, e.g. less than 9%, 8%, 7%, 6% or 5%.

In the embodiments of the present invention, in which the spherical particles (II) contain one or more active ingredients, the active ingredient content in the total weight of the spherical particles is preferably at least 70%, e.g. at least 75%, particularly preferably 80%, 81%, 82%, 83%, 84%, 85%, 86%, 87%, 88%, 89% and most preferably at least 90%, e.g. at least 91%, 92%, 93%, 94%, 95%, 96%, 97% or 98%.

The content of the first type of pellets (I) of first active ingredient or cumulative content of the first type of pellets (I) of first active ingredient and further active ingredient or further active ingredients in the uncoated state is preferably in the range from 50-100%. In exemplary embodiments, the active ingredient content is at least 60% or 75%, or at least 80%, 81%, 82%, 83%, 84%, 85%, 86%, 87%, 88%, 89% or at least 90%, e.g. at least 91%, 92%, 93%, 94%, 95%, 96%, 97%, 98%, 99% up to 100%. For example, in the case of ibuprofen a 100% active ingredient content can be realized.

Thus production and/or granulation methods and corresponding tablets are also provided for relatively high-dose active ingredients in combination with pellet products and/or covered active ingredients (I) with a relatively high active ingredient content in order to simultaneously make possible good processing, disintegration and dissolution properties of all pharmaceutically active constituents of an administration form, which during production essentially leave unchanged the characteristics of the incorporated pellets (I), especially those of dissolution.

With the aid of the present invention, especially advantageous combination preparations of two or more active ingredients in the form of tablets can be provided, in which at least one active ingredient is released in a modified manner, e.g. two active ingredients are released in a modified manner. The present invention is further particularly advantageous at a relatively high dose of the active ingredient or of the active ingredients.

In the production of these spherical particles (II), which are admixed to the pellet products (I), parts of the outer phase of a multiparticulate tablet, especially the content of microcrystalline cellulose, can be replaced by spherical particles (II), so that the phase ratio of inner phase to outer phase of this multiparticulate tablet matches that of a conventional tablet. On account of the saving of outer phase, a tablet having acceptable dimensions and acceptable swallowability can be achieved even with a high active ingredient content and in the case of combination preparations.

The spherical particles (II) produced using the method described above and the composition described above preferably have a diameter of between 5 μm and 1500 μm, especially between 50 μm and 500 μm, for example less than 400 μm, less than 300 μm, greater than 100 μm and greater than 150 μm.

The spherical aggregates (II) are preferably globular and have a sphericity of 0.8-1.0, for example of 0.85 to 1.0, from 0.9 to 1.0 and especially of 0.95 to 1.0. The sphericity is calculated here according to the following formula:

SPHT=4πA/U²

where A=area and U=circumference.

The sphericity can be carried out with apparatuses for particle size and particle shape analysis using dynamic image analysis. An apparatus suitable for this is, for example, the CAMSIZER from Retsch.

Furthermore, it is preferred that the ratio of breadth to length of the spherical aggregates (II) is in the range from 0.8 to 1.0, especially from 0.9 to 1.0. The ratio of breadth to length is calculated here according to the following formula:

b/l=min(x_{c})/Max(x_{Fe})

where x_{Fe}=Feret diameter and x_{c}=maximum breadth of the particle.

The breadth/length ratio can also be determined, for example, using the CAMSIZER from Resch.

The bulk density, determined according to the methods documented in current pharmacopeias, of these spherical aggregates produced by means of a bladed rotor is ≦0.8 g/ml, e.g. ≦0.7 g/ml, but preferably ≦0.6 g/ml and especially ≦0.5 g/ml.

The absolute porosity, that is the percentage of the total hollow cavity volume to the apparent volume, of the spherical particles (II) is preferably is preferably in a range from 0.5 to 30%, e.g. from 1 to 20%, from 1 to 10% or from 2 to 10%.

The aforementioned preferred ranges for the characteristics of the spherical particles (II) likewise apply alone or in combination as preferred for the pellets (I) used in the tablets.

After the admixing of the spherical particles (II) to give a pellet product (I) and subsequent tableting, the release behavior of the pellets (I), especially of the modified-release pellets (I), for example, changes only insignificantly or not at all compared to the non-tableted pellets, and the tableting mixture can be processed to give tablets having an acceptable weight and content uniformity and adequate mechanical strength.

Examples of active ingredients and/or active ingredient classes which can be employed advantageously in the context of the present invention are:

Medicaments for the treatment of pain and for pain therapy with peripherally acting analgesics, centrally acting analgesics and adjuvant non-analgesics. Those concerned here are the following analgesics and adjuvant substances, alone or in combination:

acetylsalicylic acid, ibuprofen, diclofenac, indomethacin, naproxen, piroxicam, paracetamol, metamizole, celecoxib, parecoxib, tramadol, pethidine, codeine, dihydrocodeine, piritramide, tilidine, morphine, hydromorphone, oxycodone, levomethadone, fentanyl, sufentanil, buprenorphine, pentazocine, naloxone, flupirtin, carbamazepine, metoprolol, metoclopramide, amitryptiline, doxepine, clomipramine, minaserine, maprotiline, triptans such as, for example, naratriptan, rizatriptan, sumatriptan, zolmitriptan, calcium antagonists such as: flunarizine, topiramate, valproic acid, phenytoin, baclofen, other agents such as: botulinum toxin, ergotamine, lisuride, methysergide, pizotifen, oxcarbazepine, gabapentine and lamotrigine, dexamethaone, methyl-prednisolone, prednisolone, triamcinolone, diazepam, tetrazepam, tizanidine, butylscopolamine, combination of tilidione and naloxone.

Pharmaceuticals/medicaments for the treatment of the nervous system, alone or in combination, e.g. seizure disorders (clonazepam, diazepam, lorazepam, midazolam, clobazam, phenytoin, clomethiazole, valproic acid, phenobarbital, gabapentin, lamotrigin, oxcarbazepine, pregabaline, topiramate, ethosuximide, levetrazetam, mesuximide, primidone, nitrazepam, vigabitrine), Parkinson syndrome (levodopa, with benserazide/carbidopa, bromocriptine, cabergoline, dihydroergocriptine, lisuride, pergolide mesilate, pramipexol, ropinirol, apomorphine, biperidene, metixene hydrochloride, trihexphenidyl, entacapone, amantadine, bupidine, selegiline, apomorphine), stroke (acetylsalicylic acid, clopidogrel, dipyridamole, ticlopidine, heparin, phenprocoumon, warfarin, protamine, phytomenadione, nimodipine, paracetamol, tramadol, buprenorphine), intracranial pressure (furosemide, mannitol), tremor (propranolol, clozapine, alprazolam, primidone).

Pharmaceuticals/medicaments, alone or in combination, for the treatment of psychiatric disorders such as anxiety disorders (alprazolam, diazepam, fluoxetin, paroxetin, chlorprothixene, levomepromazine, thioridazine, flupentixol, fluspirilene, etc.), depression (imipramine, amitripytline, desipramine, maprotiline, minaserine, citalopram, fluoxetine, paroxetine, trazodone, moclobemide, miratazepam etc.), psychoses and schizophrenia (sulpiride, promazine, melperone, thioridazine, chlorprothixene, perazine, pimozide, fluphenazine, olanzapine, risperidone, etc.), sleep disorders (triazolam, brotizolam, oxazepam, flurazepam, nitrazepam, temazepam, zolpidem tartrate, zopiclone, promethazine, chlorprothixene, pipamperone, thioridazine, chloral hydrate, etc.), states of agitation and confusion (alprazolam, oxazepam, doxepine, clomipramine, imipramine, thioridazine, perazine, etc.), dementia of the Alzheimer type (donepezil, rivastigmine, tacrin, memantine, nimodipine, seleginine etc.).

Pharmaceuticals/medicaments for the treatment of cardiovascular diseases, alone or in combination, such as coronary heart disease/angina pectoris (acetylsalicylic acid, clopidrogel, ticlopidine, isosorbide dinitrate, isosorbide mononitrate, nitroglycerine, molsidomine, trapidil, metoprolol, bisoprolol, atenolol, acebutolol, carvedilol, nitrendipine, nifedipine, diltiazem, verapamil, benazepril, lisinopril, ramipril, fosinopril, enalapril, etc.), cardiac infarction and cardiac insufficiency (isosorbide mono- and dinitrate, clopidogrel, ticlopidine, captoprol, ramipril, lisinopril, candesartan, eproartan, irbesatan, losartan, chiortalidone, xipamide, hydrochlorothiazide, furosemide, piretanide, triameterene, digitalis glycosides, carvedilol, metoprolol, prazosine, etc.), cardiac arrhythmias (ajmaline, quinidine, disopyramide, flecainide, propafenone, propranolol, carvedilol, amiodarone, verapamil, diltiazem, etc.), hypertension (metoprolol, atenolol, urapidil, clonidine, dihydralazine, chiortalidone, hydrochloroithiazide, furosemide, felodipine, israpidine, lacidipine, diltiazem, captopril, enalapril, fosinopril, lisinopril, ramnipril, verapamil, candesartan, eprosartan, irbesatan, losartan, doxazosine, bunazosine, prazosine, terazosine, moxonidine, dihydralazine, minoxidil).

Pharmaceutical/medicaments, alone or in combination, for the treatment of the airways and the lung (theophylline, methylprednisololne, flucortolne, dexamethasone, montelukast, roxithromycin, erythromycin, azithromycin, ciprofloxacin, clarithromycin, levofloxacin, ofloxacin, doxycycline, ampicillin and sulbactam, amoxicillin, cefuroxime, clindamycin, cefotiam, cefuroxime, cefotiam, ceftazidime, ceftriaxone, piperacillin, moxifloxacin, etc.).

Pharmaceutical/medicaments, alone or in combination, for the treatment of the gastrointestinal tract and of the structure salivary gland (fluconazole, mesalazine, sulfasalazine, budenoside, azathioprine, prednisone, metronidazole, infliximab, loperamide, cotrimoxazole, ciprofloxacin, metronidazole, vancomycin, esomeprazole, lansoparzole, pantoprazole, rabeprazole, cimetidine, famotidine, ranitidine, nizatidine, sucralfate, misoprostol, metoclopramide, pirenzepine, plantago seeds, bisacodyl, domperidone, sulpiride, alizapride, dimenhydrinate, cinnarizine, flunarizine, levomeprazine, ondansetron, betahsitidine, aprepitant, etc.).

Pharmaceutical/medicaments, alone or in combination, for infection defense with antibiotics or antivirally active substances (acyclovir, amantadine, azithromycin, bacampicillin, cefaclor, cefazoline, cefixime, cefprozil, ceftriaxone, chloroquine, ciprofloxacin, clotrimazole, dicloxacillin, doxycyclline, econazole, erythromycin, ethambutol, fosfomycin, flucloxacillin, fluconazole, fusidic acid, gramicidin, idoxuridine, indinavir, interferon, itraconazole, isoniazide, josamycin, ketoconazole, lamivudine, lomefloxacin, mafenide, mebendazole, mesalazine, mezlozillin, mupirocine, miconazole, naftifine, nalidixic acid, norfloxacine, ofloxacine, oxacillin, oxytetracycline, piperacillin, praziquantel, primaquim, proguanil, ribavirin, rifabutin, rimantadine, roxothromycin, saquinavir, spectinomycin, spriramycin, stavudine, sulbactam, teiucoplanin, terbinafine, tetracycline, tetroxoprim, ticarcillin, tinidazole, tromantadine, tolnaftate, vancomycin, zidovudine, zalcitabine, etc.).

Pharmaceuticals/medicaments for the treatment of erectile dysfunction, alone or in combination (sildenafil, tadalafil, vardenafil, theobromine, caffeine, theophylline, etc.).

Combination preparations are especially combinations of active ingredients for the treatment of cardiovascular diseases, for instance high blood pressure, for the treatment of the central nervous system (CNS), for instance Parkinson's disease or depression, for infection defense with antibiotics or antivirally active substances, for oral contraception, for the treatment of gastric diseases and in pain therapy.

The tablet can be, for example, an orally dispersible tablet. Alternatively, the tablet, also conventionally formulated, can disintegrate into its subunits only in lower sections of the gastrointestinal tract.

The invention is described below with reference to a number of exemplary embodiments and the following figures. These show

FIG. 1 the percentage mass fractions of the inner and outer phase of conventional, multiparticulate tablets and of tablets for the purposes of this invention;

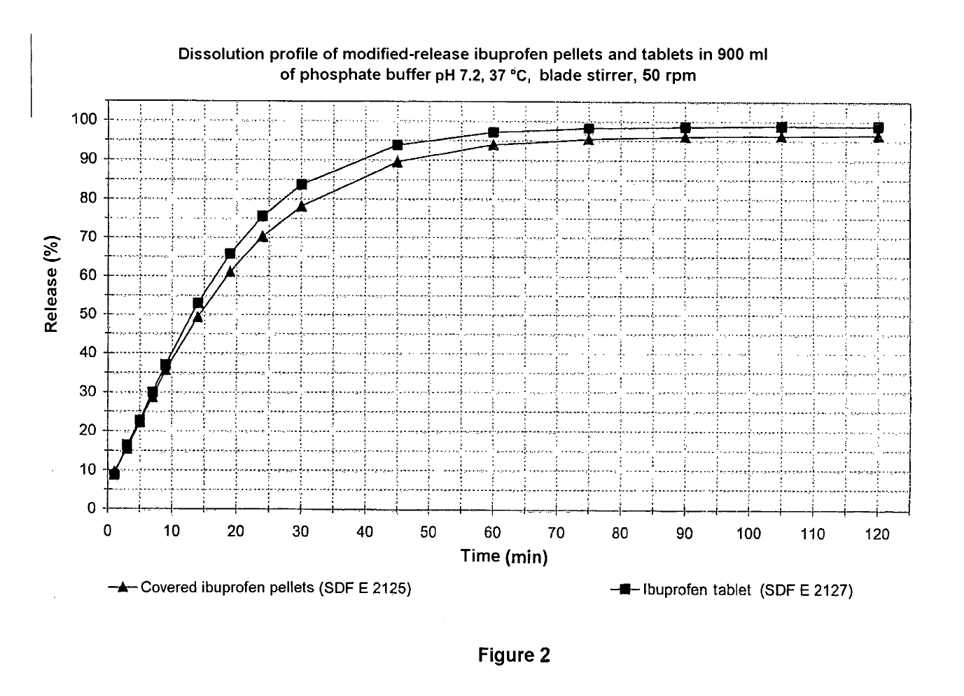

FIG. 2 the dissolution profile of a tablet according to a first exemplary embodiment of the invention in comparison to the dissolution profile of the pellets used; and

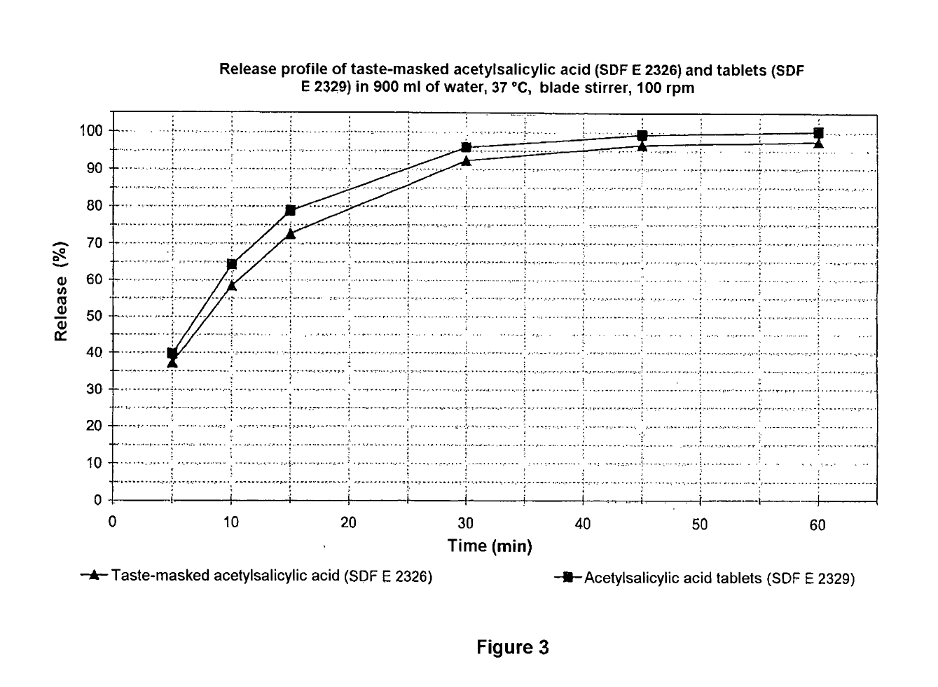

FIG. 3 the dissolution profile of a tablet according to a second exemplary embodiment of the invention in comparison to the dissolution profile of the pellets used;

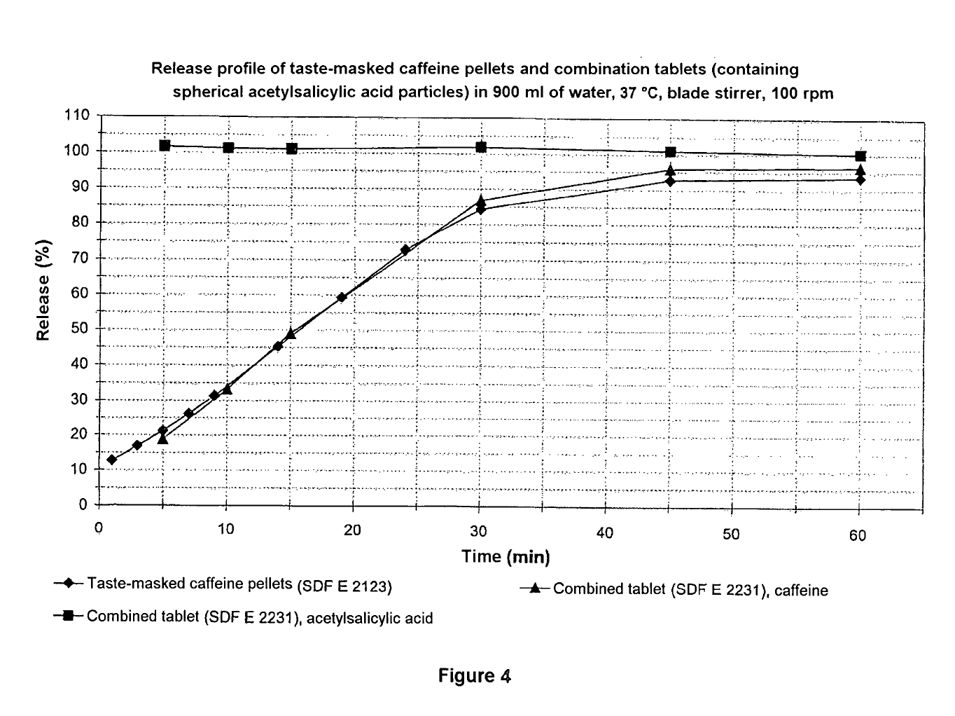

FIG. 4 the dissolution profile of a tablet according to a third exemplary embodiment of the invention in comparison to the dissolution profile of the pellets used.

### EXAMPLE 1

Modified-release ibuprofen tablets

Single dose

| Component | Mass (mg) | Content (%) |
|---|---|---|
| Pellet | | |
| Ibuprofen pellets | 200.0 | 28.57 |
| Delaying film coating | | |
| Ethocel Standard 10 Premium | 30.0 | 4.29 |
| HPC-L | 30.0 | 4.29 |
| Spherical particles | | |
| Pearlitol 25 C | 391.3 | 55.90 |
| Kollidon CL-M | 43.5 | 6.21 |
| outer phase | | |
| Sodium stearylfumarate | 5.3 | 0.75 |
| Total | 700.00 | 100.00 |

300-1000 g of ibuprofen pellets of particle size 100-200 μm are coated in a fluidized bed apparatus having a Wurster insert (GPCG 1, Glatt, having a 6″ Wurster insert) with a solution containing 2.5% of Ethocel Standard 10 Premium from Colorcon and 2.5% of HPC-L from Nisso in ethanol at a product temperature of 30° C. and a spray rate of approximately 5-10 g/min.

For the production of the spherical particles, Kollidon CL-M from BASF and Pearlitol 25 C from Roquette are mixed in the mass ratio 10:90 in a rapid mixer, VG 10, Glatt, and homogeneously premoistened with a 15% solution of Pearlitol 160 C in water. The residual moisture of this product is approximately 10% (Mettler halogen dryer, approximately 5 g, 105° C., 1 mg/30 sec). Subsequently, the premoistened material is spheronized with addition of demineralized water in the bladed rotor to give spherical particles, until the mean grain size is approximately 200 μm. The bladed rotor, CPS 3, Glatt, is equipped for this purpose with a 30° rotor plate. In the processing space are located four flat blades, in order to align the material movement accordingly. The inlet air temperature during the spheronization is 35° C. The quantity of air approximately 75 m³/h. The rotor speed is 350 rpm. The residual moisture of this material is 20%, determined according to the method indicated above. The spherical particles thus obtained are subsequently dried in the fluidized bed at an inlet air temperature of 60° C. until the residual moisture of the dried product reaches a value of approximately <0.2%. The bulk density of these spherical particles is 0.6 g/ml.

The coated ibuprofen pellets are mixed in the ratio indicated above with the spherical particles consisting of Pearlitol 25 C and Kollidon CL-M and the sodium stearylfumarate (Pruv, JRS) in the Turbula T2C (Bachofen).

Subsequently, tablets having a diameter of 16 mm are produced, using a tablet press, e.g. Korsch EK 0. The hardness of the tablets is 30 N. The release behavior of the coated pellets and the tablet is reproduced in FIG. 2.

### EXAMPLE 2

Taste-masked acetylsalicylic acid tablets

| | |
|---|---|
| Component | Single dose |

| Active ingredient | Mass (mg) | Content (%) |
|---|---|---|
| Acetylsalicyclic acid | 250.0 | 41.67 |
| Taste-masking coating | | |
| Ethocel Standard 10 Premium | 30.0 | 5.00 |
| HPC-L | 7.5 | 1.25 |
| Spherical particles | | |
| Pearlitol 25 C | 262.4 | 43.73 |
| Kollidon CL-M | 29.2 | 4.86 |
| outer phase | | |
| Aerosil 200 | 3.0 | 0.50 |
| Lubritab | 18.0 | 3.00 |
| Total | 600.00 | 100.00 |

750 g of acetylsalicylic acid is sprayed into the fluidized bed with a taste-masking covering of 80% Ethocel Standard 10 Premium (Colocon) and 20% HPC-L (Nisso) into the fluidized bed in the topspray method at a product temperature of 30° C. The components (Ethocel and HPC) are dissolved in ethanol. The solid content of this spray solution is 5%.

For the production of these spherical particles, Kollidon CL-M from BASF and Pearlitol 25 C from Roquette are mixed in the mass ratio 10:90 in the rapid mixer VG 10, Glatt, and homogeneously premoistened with a 15% strength solution of Pearlitol 160 C in water. The residual moisture of this product is approximately 10% (Mettler halogen dryer, approximately 5 g, 105° C., 1 mg/30 sec). Subsequently, the premoistened material is spheronized with addition of demineralized water in the bladed rotor to give spherical particles, until the mean particle size is approximately 200 μm. The bladed rotor, CPS 3, Glatt, is equipped for this purpose with a 30° rotor plate. In the processing space are located four flat blades, in order to align the material movement accordingly. The inlet air temperature during the spheronization is 35° C. The quantity of air approximately 75 m³/h. The rotor speed is 350 rpm. The residual moisture of this material is approximately 20%, determined according to the method indicated above. The pellets are subsequently dried in the fluidized bed at an inlet air temperature of 60° C. until the residual moisture of the dried product reaches approximately <0.2%. The bulk density of these spherical particles is 0.6 g/ml.

The coated acetylsalicylic acid is mixed in the Turbula T2C (Bachofen) in the ratio indicated above with the spherical particles consisting of Pearlitol 25 C and Kollidon CL-M and Lubritab (JRS) and Aerosil 200 (Evonik).

Subsequently, tablets with a diameter of 13 mm are produced using a tablet press, e.g. Korsch EK 0. The hardness of the tablets is 30 N. The release behavior of the coated acetyl salicylic acid and the tablet produced therefrom is reproduced in FIG. 3.

### EXAMPLE 3

Acetylsalicylic acid/caffeine tablet

Single dose

| Component | Mass (mg) | Content (%) |
|---|---|---|
| Caffeine pellets | | |
| Caffeine | 50.0 | 8.82 |
| Pearlitol 25 C | 14.3 | 2.52 |
| Kollidon K 30 | 7.1 | 1.25 |
| Taste-masking coating | | |
| Ethocel Standard 10 Premium | 17.1 | 3.02 |
| HPC-L | 4.3 | 0.76 |
| Spherical acetylsalicylic | | |
| acid particles | | |
| Acetylsalicylic acid | 250.0 | 44.09 |
| Pearlitol 25 C | 73.3 | 12.93 |
| Kollidon K 30 | 37.5 | 6.61 |
| External phase | | |
| Kollidon CL | 56.7 | 10.00 |
| Pearlitol 160 C | 49.6 | 8.75 |
| Aerosil 200 | 2.8 | 0.50 |
| Sodium stearylfumarate | 4.3 | 0.75 |
| Total | 567.0 | 100.00 |

In a rapid mixer, caffeine, Pearlitol 25 C (Roquette), Kollidon K 30 (BASF) are mixed and premoistened with an aqueous solution of Kollidon K 30 (10%) and Pearlitol 25 C (10%). The residual moisture of the material is 6% (Mettler halogen dryer, 105° C., 5 g, 1 mg/30 sec). The mass premoistened in this way is rounded in a bladed rotor with simultaneous spraying with water to give pellets. The bladed rotor, CPS 3, Glatt, is equipped for this purpose with a 45° rotor plate. In the processing space are situated four flat blades, in order to align the material movement accordingly. The inlet air temperature during the spheronization is 35° C. The quantity of air approximately 30 m³/h. The rotor speed 800+/−100 rpm. The caffeine pellets thus formed are dried at 60° C. inlet air temperature in the fluidized bed (Glatt, GPCG 1). The residual moisture of the material is 0.4%. The mean grain size. determined by means of screen analysis, is approximately 200 μm.

For taste masking, 300-1000 g of caffeine pellets are coated at a product temperature of 30° C. with a taste-masking coating of 80% Ethocel Standard 10 Premium (Colorcon) and 20% HPC-L (Nisso) in the fluidized bed with a Wurster insert (GPCG1, Glatt, 6″ Wurster). The components (Ethocel and HPC) are dissolved in ethanol. The solids content of the solution is 5%.

Acetylsalicylic acid, Pearlitol 25 C, Kollidon K 30 are mixed in a rapid mixer and premoistened with an aqueous solution of Kollidon K 30 (10%) and Pearlitol 25 C (10%). The premoistened mass thus obtained is rounded in a bladed rotor with simultaneous spraying with water to give spherical particles. The bladed rotor, CPS 3, Glatt, is equipped for this purpose with a 30° rotor plate. In the processing space are situated four flat blades, in order to align the material movement accordingly. The inlet air temperature during the spheronization is 35° C. The quantity of air approximately 50 m³/h. The rotor speed is 500 rpm. The acetylsalicylic acid pellets thus obtained are dried at 60° C. inlet air temperature in the fluidized bed (Glatt, GPCG 1). The residual moisture of the material is <0.2%. The mean grain size, determined by means of screen analysis, is approximately 125 μm. The bulk density is approximately 0.5 g/ml.

The coated caffeine pellets are mixed in the Turbula T2C (Bachofen)) in the ratio indicated above with the spherical acetylsalicylic acid particles, which in addition to the active ingredient consist of Pearlitol 25 C (Roquette) and Kollidon K 30 (BASF), and the components of the outer phase (Kollidon CL (BASF), Pearlitol 160 C (Roquette), Aerosil 200 (Evonik) and sodium stearylfumarate (Pruv, JRS).

Subsequently, tablets having a diameter of 13 mm tablets are produced using a tablet press, e.g. Korsch EK 0. The hardness of the tablets is 30 N. The release behavior of the coated caffeine pellets and the tablets resulting therefrom is reproduced in FIG. 4.

1. A tablet, containing
an inner phase, comprising
a) a first type of pellets (I), which contain a first active ingredient and which have a coating and/oder release the active ingredient in a modified manner, or
b) a first type of covered first active ingredient (I), mixed with
c) spherical particles (II),
and an outer phase containing one or more excipients present in non-granulated form before tableting, selected from disintegrants, lubricants, flow regulators, fillers and binders,
wherein the weight of the outer phase makes up not more than 25 percent of the total weight of the tablet.

2. The tablet as claimed in claim 1,
wherein the first type of pellets (I) is covered with a film which modifies the release of the first active ingredient from the pellets (I).

3. The tablet as claimed in claim 1,
wherein the coating of the first active ingredient is a coating which modifies the dissolution of the active ingredient.

4. The tablet as claimed in one of the preceding claims, wherein the spherical particles (II) contain a second active ingredient.

5. A tablet containing:
a first type of pellets (I), which contain a first active ingredient and which have a coating and/or release the active ingredient in a modified manner, or a first type of covered first active ingredient (I),
mixed with spherical particles (II), the difference in the dissolution profiles of the first type of pellets (I) after admixing the spherical particles (II) and subsequent tableting being less than 20%.

6. A tablet containing:
an inner phase, comprising spherical particles (II) which contain an active ingredient,
and an outer phase containing one or more excipients present in non-granulated form before tableting, selected from disintegrants, lubricants, flow regulators, fillers and binders,
wherein the weight of the outer phase makes up not more than 25 percent of the total weight of the tablet, for example not more than 20%, 17.5%, 15%, 12.5%, 10%, 7.5% or 5%.

7. The tablet as claimed in claim 6, wherein the proportion of the active ingredient in the weight of the spherical particles (II) is at least 80%.

8. The tablet as claimed in one of the preceding claims, wherein the spherical particles (II) have a density of less than 0.8 g/ml.

9. The tablet as claimed in one of the preceding claims, wherein the spherical particles (II) are produced by means of a moist granulation method.

10. The tablet as claimed in claim 9, wherein the spherical particles are produced by means of rotor granulation.

11. The tablet as claimed in claim 10, wherein the spherical particles are produced using a bladed rotor.
